(19) United States
(12) Patent Application Publication
(10) Pub. No.: US 2008/0216829 A1
(43) Pub. Date: Sep. 11, 2008

(54) PROCESS FOR REGULATING A RESPIRATION HUMIDIFIER AS WELL AS A CORRESPONDING DEVICE

(75) Inventors: Jochim KOCH, Ratzeburg (DE); Klaus RADOMSKI, Lubeck (DE); Thomas ROSSEN, Lubeck (DE)

Correspondence Address: MCGLEW & TUTTLE, PC, P.O. BOX 9227, SCARBOROUGH STATION, SCARBOROUGH, NY, 10510-9227, US

(73) Assignee: DRAGER MEDICAL AG & CO. KG, Lubeck (DE)

(21) Appl. No.: 12/028,977

(22) Filed: Feb. 11, 2008

(30) Foreign Application Priority Data
Mar. 9, 2007 (DE) 10 2007 011 544.1

(51) Int. Cl. A61M 16/16; G01N 27/00

(52) U.S. Cl. 128/203.26; 73/335.02

(57) ABSTRACT

A process and a device are provided for regulating a respiration humidifier by the temperature measured in the breathing gas. The output of the heating or dispensing unit (15), which releases its moisture into a mixing chamber (11), to which breathing gas flowing in is admitted, is set as a function of the dew coating measured downstream of the mixing chamber (11) such that an electrical resistance or a capacitance measured in the breathing gas downstream of the mixing chamber (11) between at least two measuring points (4, 5) as an indicator of dew coating is in a preset range of desired values with a maximum and a minimum.

### CROSS REFERENCE TO RELATED APPLICATIONS

This application claims the benefit of priority under 35 U.S.C. §119 of German Patent Application DE 10 2007 011 544.1 filed Mar. 9, 2007, the entire contents of which are incorporated herein by reference.

### FIELD OF THE INVENTION

The present invention pertains to a process for regulating a respiration humidifier as well as to a device for carrying out the process.

### BACKGROUND OF THE INVENTION

It is necessary, especially for the artificial respiration of patients, to humidify the breathing gas being fed to the patient being respirated by corresponding respiration humidifiers such that the patient will not dry out and is exposed to physiologically pleasant respiration conditions as much as possible. In general, condensation or excessive humidifying shall be avoided during the operation of such respiration humidifiers. In most respiration humidifier designs, the starting conditions of the humidifier are checked immediately downstream of the mixing chamber by measuring the temperature in the humidified breathing gas. This parameter is also used, in general, to regulate the humidifier. However, if the temperature sensor is wetted with moisture or is even coated with droplets, it may happen that the temperature sensor will cool due to the evaporation and measures temperatures that are too low. In case of a correspondingly regulated humidifier design, this can consequently cause even more water or moisture to be fed because of the temperature drop, so that humidification escalates and may lead to an undesired generation of condensate in the breathing tube system.

Overdosage of moisture in the respiration tube system may also occur in prior-art respiration humidifiers with an evaporation chamber, because the initial temperature of the evaporation chamber is adjusted to the desired value of the breathing gas temperature. If the outlet temperature at the temperature sensor is distorted by the evaporative cooling of condensed water droplets and this sensor correspondingly displays excessively low temperatures, the heat output is increased and more moisture is generated, which will then condense in the breathing tube system or at the latest in the patient's tube, which should be avoided by all means.

It is known from DE 100 38 365 C2 that a temperature sensor in an evaporation chamber is protected from condensate drops by the use of a baffle plate.

In many cases, such an arrangement cannot be embodied, on the one hand, because of construction due to lack of sufficient space; on the other hand, respiration shall not be interfered with, and drop formation at the temperature sensor cannot be prevented from occurring by flow swirling. When the surface temperature of the temperature sensor drops below the dew point, dew coating of the sensor cannot be physically avoided.

Heating of the temperature sensor or of the carrying housing cannot be considered because the exact breathing gas temperature shall be determined.

### SUMMARY OF THE INVENTION

The object of the present invention is to provide an improved process and a device for regulating a respiration humidifier so that overmoistening, but also undermoistening can be reliably prevented from occurring.

According to the invention, a process is provided for regulating a respiration humidifier by means of the temperature measured in the breathing gas. The process comprises admitting a breathing gas flow into a mixing chamber and releasing moisture into a mixing chamber, with a heating or dispensing means. The moisture is released as a function of a dew coating measured downstream of the mixing chamber such that an electrical characteristic measured in the breathing gas downstream of the mixing chamber at a location between at least two measuring points as an indicator of the dew coating is in a preset range of desired values with a maximum and a minimum.

The minimum heating or dispensing output may be limited by a preset limit value of the electrical characteristic measured and the electrical characteristic measured may be the electrical resistance or the capacitance corresponding to a minimum moisture content in the breathing gas. The maximum heating or dispensing output may be limited corresponding to a maximum breathing gas moisture content by a preset limit value of the electrical resistance or of the capacitance as the electrical characteristic measured.

According to another aspect of the invention, a device is provided for regulating a respiration humidifier by means of the temperature measured in the breathing gas. The device comprises a mixing chamber with a breathing gas inlet and a humidified breathing gas outlet, a heating or dispensing means releasing moisture into the mixing chamber and a measuring unit arranged in a path of the humidified breathing gas downstream of the mixing chamber. The measuring unit has a temperature sensor and has at least two measuring point contacts for a determination of an electrical characteristic as an indicator of a dew coating. A measuring and regulating unit is connected to the heating or dispensing means and to the measuring unit for regulating the heating or dispensing means based on measurements made by the measuring unit.

The measuring unit may have a housing made of an electrically insulating plastic. The plastic may have an electrical resistance or capacitance in the breathing gas flow that depends on the humidity of the breathing gas flow and is used as the electrical characteristic measured.

An inspiration tube is connected to the breathing gas outlet, wherein the measuring unit is arranged in the inspiration tube. The inspiration tube may include a Y-piece and the measuring unit may be arranged at or in front of the Y-piece. The inspiration tube may be provided with a heater, which is controlled by the measuring and regulating unit.

The dispensing means releasing moisture may be a water evaporator or a membrane humidifier. The respiration humidifier may advantageously be connected to an anesthesia apparatus or a respirator.

According to another aspect of the invention, a process is provided for breathing gas comprising the steps of providing a mixing chamber with a breathing gas inlet and a humidified breathing gas outlet, providing a heating or dispensing means and arranging a measuring unit in a path of the humidified breathing gas downstream of the mixing chamber. The measuring unit has a temperature sensor and has at least two measuring points (electrical contacts) for a determination of an electrical resistance or for a determination of a capacitance as an indicator of a dew coating. A measuring and regulating unit is connected to the heating or dispensing means and to the measuring unit for regulating the heating or dispensing means based on measurements made by the measuring unit. Breathing gas flow is admitted into the mixing chamber for flow from the inlet to the outlet. A respiration humidity is controlled with the measuring and regulating unit based on the temperature measured in the breathing gas by releasing moisture into a mixing chamber, with the heating or dispensing means, as a function of a dew coating measured at the measuring points downstream of the mixing chamber such that the electrical resistance or the capacitance measured in the breathing gas downstream of the mixing chamber between at least two measuring points as an indicator of the dew coating is controlled to be in a preset range of desired values with a maximum value and a minimum value.

The essential advantage of the process in accordance with the invention is that the surface of the temperature sensor or of the housing with the temperature sensor is used as a sensor system for the electrical measurement of the surface moisture or dew coating.

The housing or the measuring unit is provided for this purpose especially with two measuring points designed at electric contacts, between which the electrical characteristic such as the electric resistance, the electrical conductivity or even the capacitance (capacitive reactance) is measured.

If the housing of the temperature sensor consists, for example, of a plastic having poor electrical conductivity, the surface has a very high electrical resistance in the dry state. The resistance drops to a few MOhms (MΩ) at a humidity of about 95% relative humidity at 37° C. If drops of moisture are formed between the contacts, the electrical resistance drops markedly to a fraction of a few MΩ.

The electrical resistance measurement can also be integrated in the surface of the housing of the measuring unit in a relatively simple manner by leading the contacts to the surface from the inside, so that the wiring proper is located in the housing and is protected. Cleaning of the measuring unit and of the temperature sensor is possible without problems.

The measurement of the electrical characteristic—of the electrical resistance, or conductance or, alternatively, of the capacitance—can be implemented in a simple manner, at a low cost and in a reliable manner.

To enlarge the contacts on the surface, parts of the housing surface of the measuring unit may be provided with electrically conductive partial areas, for example, in the form of rings about the cylindrical measuring unit or by means of vapor-deposited conductive surfaces.

As an alternative, the measurement of the condensed moisture or dew cover may also be carried out by measuring the electrical capacity between the measuring points designed as electrical contacts, as this is known per se in connection with moisture sensors.

If a measuring unit as described is available for a respiration humidifier, the output signal can be used to regulate the humidification.

If, for example, the surface of the temperature sensor or the housing of the measuring unit is wetted with moisture and the measured electrical resistance decreases correspondingly, the quantity of water released into the breathing gas flow can be reduced in case of a metered humidifier until the resistance again rises to a preset desired value and wetting with water has decreased.

In a respiration humidifier with a dispensing means designed as an evaporation chamber, the output signal can be used to reduce the heat output or the evaporator chamber temperature at the hot plate to the extent that the moisture at the outlet of the mixing chamber will enter ranges of values that prevent condensation from occurring.

The process described is also suitable for reliably avoiding an excessively dry humidification. The regulation of a respiration humidifier, which uses only the temperature at the outlet of or downstream of the mixing chamber, cannot determine whether the moistened breathing gas is undersaturated or supersaturated.

Undersaturation of the breathing gas may lead to discomfort for the patient over time. Thus, it is specified by ISO 8185 that respiration humidifiers shall supply a minimum moisture content of 75% relative humidity corresponding to 33 mg/L of water in air.

The regulation of the respiration humidifier can be carried out here such that the measured electrical resistance at the measuring unit is maintained within a range of desired values.

Both condensation, i.e., overmoistening, and undermoistening can be reliably avoided with the process being described.

An exemplary embodiment of the present invention will be explained below on the basis of the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which the preferred embodiment of the invention is illustrated.

### BRIEF DESCRIPTION OF THE DRAWINGS

In the drawings:

FIG. 1 is a block diagram of a respiration humidifier; and

FIG. 2 is a schematic view of the measuring unit used.

### DESCRIPTION OF THE PREFERRED EMBODIMENT

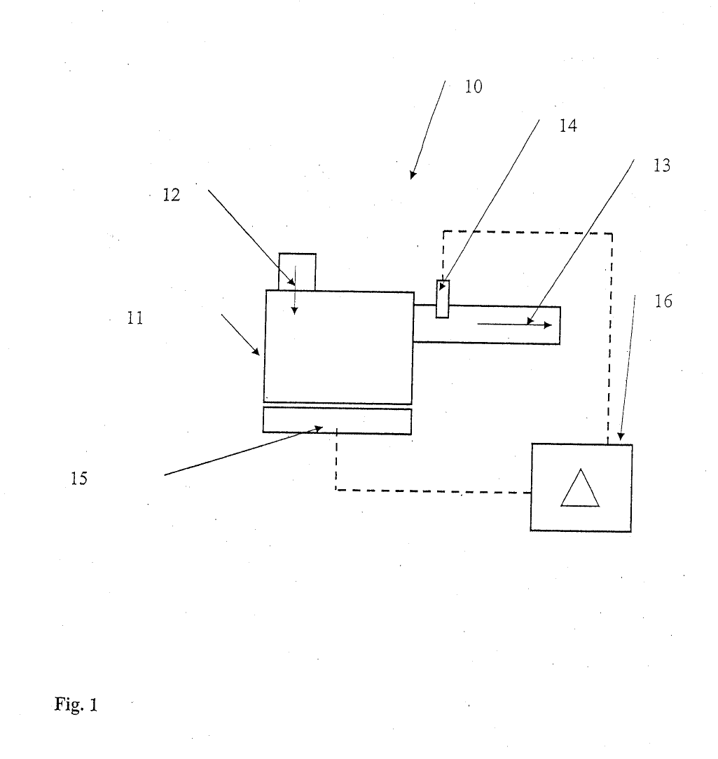

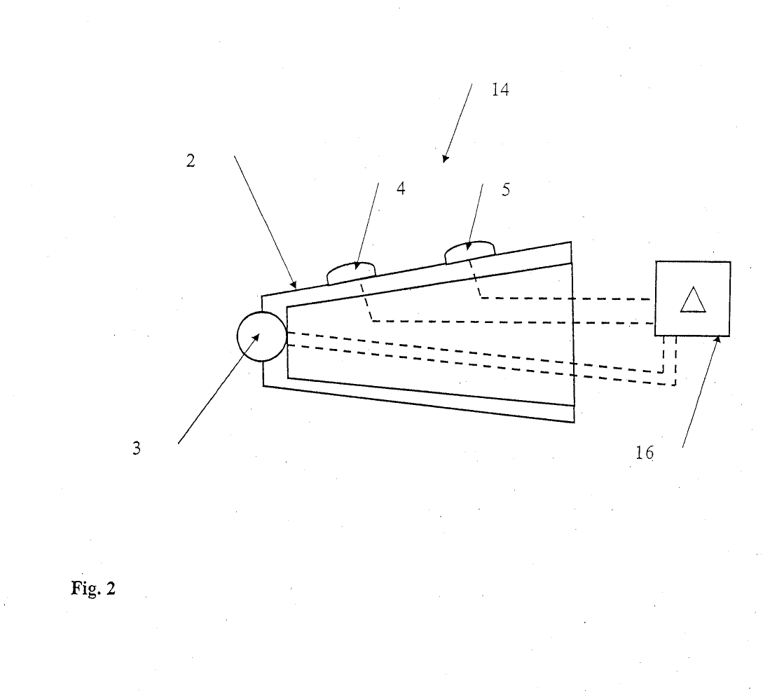

Referring to the drawings in particular, the respiration humidifier 10 in FIG. 1 is regulated by means of the measuring and regulating unit 16 as a function of the temperature measured in the moistened breathing gas. Moisture, which is released by a heating or dispensing means 15 into the mixing chamber 11, is fed in the mixing chamber 11 to the breathing gas flowing in via the breathing gas feed unit 12 in the direction of the arrow. The moistened breathing gas is again discharged from the mixing chamber 11 via the breathing gas discharge line 13 and flows around the measuring unit 14 with the temperature sensor 3, see FIG. 2. If the measuring unit 14 or its housing 2 is wetted with moisture, this is recorded by means of the measuring and regulating unit 16 and the electric output of the heating means 15 is correspondingly reduced. When the actual value drops below a preset desired value of the moisture measured at the measuring unit 14 on the basis of the electrical resistance, the heat output is again increased somewhat without condensation taking place at the measuring unit 14.

The respiration humidifier 10 can be regulated continuously between these two desired values.

FIG. 2 shows the design of the measuring unit 14, shown in a sectional view. The temperature sensor 3 for the measurement of the breathing gas temperature is arranged at a housing 2 made of a preferably temperature-dependent plastic having poor conductivity, especially at the tip of the housing 2 pointing in the direction of the breathing gas flowing in. Two measuring points 4, 5, which are designed as electrical contacts and between which the electrical resistance or, as an alternative, the conductivity is measured and can be sent to the measuring and regulating unit 16 for analysis, are arranged on the surface of the housing 2. The electrical resistance between the two measuring points 4, 5 decreases in case of wetting of the outer surface of the housing 2 and is used to regulate the respiration humidifier 10.

When the respiration humidifier 10 is started up, it starts out from a desired value of the moisture, which was set by the user, for example, from a moisture of 95% relative humidity. In addition, the user sets a desired breathing gas temperature downstream of the mixing chamber 11 T_{desired}, e.g., 37° C. In particular, a moisture sensor, which measures 0% relative humidity in the example when the respirator is operated especially with gas from a central supply system, is located in the inlet of the mixing chamber 11. The absolute quantity of water, X_{absolute quantity}, which is 0 mg of water per L of breathing gas in this case, is calculated from the measured value of the relative humidity and the measured temperature of the temperature sensor 3. The flow (respiratory minute volume) of the respirator V is measured; it equals 10 L/minute. The measuring and regulating unit 16 calculates from this a necessary vapor temperature of 286° C., to which the heating means 15 is regulated. The necessary quantity of water is calculated from the input values of the mixing chamber 11 and the desired value preset by the user; it equals 445 mg/minute in this example.

After the heating means 15 has been started up and the water has been fed in the form of hot steam, the breathing gas temperature T_{desired} increases to the desired value of 37° C. Based on the effects of the different tolerances of the transducers, the relative humidity is, however, too high and the gas is supersaturated, so that condensation already begins at the temperature sensor 3 of the mixing chamber outlet. The dew coating sensor arranged there between the measuring points 4, 5, which still had previously an electrical resistance approaching infinity in the dry state during start-up, slowly reduces its resistance and reaches about 3.2 MΩ at 95% relative humidity. The resistance would decrease further in the direction of 1.2 MΩ, at which droplets would already have formed on the housing 2, as the condensate formation increases at the temperature sensor 3 or at the housing 2. The internal desired value for the water feed is reduced at the latest beginning from a resistance of about 3.2 MΩ in order to avoid incipient condensation. The quantity of water is again increased somewhat when the resistance changes again in the direction of infinity. The quantity of water is regulated between these values directly via the dew coating sensor when neither the desired values nor the input conditions have changed.

In a second example, a desired value for the relative humidity of 85% is selected, which is markedly below the condensation limit. The dew coating sensor would have a resistance of about 10 MΩ. The quantity of water released by the heating or dispensing means 15 is increased briefly, e.g., for a minute, by about 10%. The relative humidity at the mixing chamber outlet now increases and the dew coating sensor at the temperature sensor 3 slowly begins to become coated with condensate. The resistance value drops to 1.2 MΩ when the relative humidity has a value of about 95%. The quantity of water is then recalculated, namely corrected downward with a factor of C=85/95 (85%=desired value, 95%=measured value). The relative humidity changes again due to the reduction of the quantity of water after about one minute to the desired value of 85%, as calculated. It is therefore possible with this process to also regulate lower humidity levels by means of the dew coating sensor without dew coating occurring at the temperature sensor 3 over the long run. The respiration humidifier 10 can be regularly “calibrated” due to the brief slight dew coating, so that tolerances of the individual sensors, which lead to different moisture contents, are compensated with this process. Such a “calibration” can be repeated depending on the operating conditions, namely, on whether they were constant or variable over a longer time.

This process can also be applied to other moistening concepts, such as cascade humidifiers (pass-over). Only the desired value of the temperature of the heating means 15 is increased or decreased in this case, depending on the direction in which the dew coating takes place at the sensor.

While specific embodiments of the invention have been shown and described in detail to illustrate the application of the principles of the invention, it will be understood that the invention may be embodied otherwise without departing from such principles.

What is claimed is:

1. A process for regulating a respiration humidifier by means of a temperature measured in a breathing gas, the process comprising the steps of:
admitting a breathing gas flow into a mixing chamber;
releasing moisture into a mixing chamber, with a heating or dispensing means, as a function of a dew coating measured downstream of the mixing chamber such that an electrical characteristic measured in the breathing gas downstream of the mixing chamber at a location between at least two measuring points as an indicator of the dew coating is in a preset range of desired values with a maximum and a minimum.

2. A process in accordance with claim 1, wherein the minimum heating or dispensing output is limited by a preset limit value of the electrical characteristic measured and the electrical characteristic measured is an electrical resistance or an capacitance corresponding to a minimum moisture content in the breathing gas.

3. A process in accordance with claim 1, wherein the maximum heating or dispensing output is limited corresponding to a maximum breathing gas moisture content by a preset limit value of an electrical resistance or capacitance as the electrical characteristic measured.

4. A device for regulating a respiration humidifier by means of a temperature measured in a breathing gas, the device comprising:
a mixing chamber with a breathing gas inlet and a humidified breathing gas outlet;
a heating or dispensing means releasing moisture into said mixing chamber;
a measuring unit arranged in a path of humidified breathing gas downstream of said mixing chamber, said measuring unit having a temperature sensor and having at least two measuring point contacts for a determination of an electrical characteristic as an indicator of a dew coating; and
a measuring and regulating unit connected to said heating or dispensing means and to said measuring unit for regulating said heating or dispensing means based on measurements made by said measuring unit.

5. A device in accordance with claim 4, wherein said measuring unit has a housing made of an electrically insulating plastic and wherein the insulating plastic has an electrical resistance or capacitance that depends on the humidity and is used as the electrical characteristic measured.

6. A device in accordance with claim 4, further comprising an inspiration tube connected to said breathing gas outlet, wherein said measuring unit is arranged in the inspiration tube.

7. A device in accordance with claim 6, wherein said inspiration tube includes a Y-piece and said measuring unit is arranged at or in front of the Y-piece, and said inspiration tube is provided with a heater, which is controlled by said measuring and regulating unit.

8. A device in accordance with claim 4, wherein said dispensing means is a water evaporator or a membrane humidifier.

9. A device in accordance with claim 4, wherein the respiration humidifier is connected to an anesthesia apparatus or a respirator.

10. A process for breathing gas comprising the steps of:
providing a mixing chamber with a breathing gas inlet and a humidified breathing gas outlet;
providing a heating or dispensing means;
arranging a measuring unit in a path of humidified breathing gas downstream of the mixing chamber wherein the measuring unit has a temperature sensor and has at least two measuring points for a determination of an electrical resistance or for a determination of a capacitance, measured between the at least two measuring points, as an indicator of a dew coating;
providing a measuring and regulating unit connected to said heating or dispensing means and to said measuring unit for regulating said heating or dispensing means based on measurements made by said measuring unit;
admitting a breathing gas flow into the mixing chamber for flow from the inlet to the outlet;
regulating respiration humidity with the measuring and regulating unit based on a temperature measured in the breathing gas by releasing moisture into a mixing chamber, with the heating or dispensing means, as a function of a dew coating measured at the measuring points downstream of the mixing chamber such that the electrical resistance or the capacitance measured in the breathing gas downstream of the mixing chamber between at least two measuring points as an indicator of the dew coating is controlled to be in a preset range of desired values with a maximum value and a minimum value.

11. A process in accordance with claim 10, wherein a minimum heating or dispensing output is limited by the preset limit value of the electrical resistance or of the capacitance corresponding to a minimum moisture content in the breathing gas.

12. A process in accordance with claim 10, wherein a maximum heating or dispensing output is limited corresponding to a maximum breathing gas moisture content by a preset limit value of the electrical resistance or of the capacitance.

13. A process in accordance with claim 10, wherein said measuring and regulating unit has a housing made of an electrically insulating plastic and wherein the insulating plastic has an electrical resistance or capacitance that depends on the humidity.

14. A process in accordance with claim 10, further comprising providing an inspiration tube connected to said breathing gas outlet, wherein said measuring and regulating unit is arranged in the inspiration tube.

15. A process in accordance with claim 14, wherein said inspiration tube includes a Y-piece and said measuring unit is arranged at or in front of the Y-piece, and said inspiration tube is provided with a heater, which is controlled by said measuring and regulating unit.

16. A process in accordance with claim 10, wherein said dispensing means is a water evaporator or a membrane humidifier.

17. A process in accordance with claim 10, wherein the respiration humidifier is connected to an anesthesia apparatus or a respirator.
